(19) United States
(12) Patent Application Publication
(10) Pub. No.: US 2018/0226094 A1
(43) Pub. Date: Aug. 9, 2018

(54) Ionic Liquid, Lubricant, and Magnetic Recording Medium

(71) Applicant: Dexerials Corporation, Shinagawa-ku, Tokyo (JP)

(72) Inventors: Hirofumi Kondo, Tokyo (JP); Kouki Hatsuda, Tokyo (JP); Nobuo Tano, Tokyo (JP)

(21) Appl. No.: 15/750,323

(22) PCT Filed: Aug. 3, 2016
(86) PCT No.: PCT/JP2016/072755

§ 371 Date: Feb. 5, 2018

(30) Foreign Application Priority Data
Aug. 6, 2015 (JP) 2015-156293

(51) Int. Cl. G11B 5/725; G11B 5/71; C10M 105/72; C10M 105/70; C07D 233/58; C07C 309/06

(52) U.S. Cl. CPC G11B 5/725; G11B 5/71; C07C 309/06; C10M 105/70; C07D 233/58; C10M 105/72

(57) ABSTRACT

A lubricant which includes an ionic liquid including a conjugate acid and a conjugate base having 2 or more anions in a molecule of the conjugate base, wherein the conjugate acid has a group including a straight-chain hydrocarbon group having 6 or more carbon atoms, and a pKa of an acid that is a source of the conjugate base in acetonitrile is 10 or less.

### TECHNICAL FIELD

The present invention relates to an ionic liquid, a lubricant including the ionic liquid, and a magnetic recording medium using the lubricant.

### BACKGROUND ART

Conventionally, in a thin film magnetic recording medium, a lubricant is applied onto a surface of a magnetic layer for the purpose of reducing frictions between a magnetic head and the surface of the magnetic recording medium, or reducing abrasion. In order to avoid adhesion, such as sticktion, an actual film thickness of the lubricant is of a molecular order. Accordingly, it is not exaggeration to say that the most important thing for a thin film magnetic recording medium is to select a lubricant having excellent abrasion resistance in any environment.

During a life of a magnetic recording medium, it is important that a lubricant is present on a surface of the medium without causing desorption, spin-off, and chemical deteriorations. Making the lubricant present on a surface of a medium is more difficult, as the surface of the thin film magnetic recording medium is smoother. This is because the thin film magnetic recording medium does not have an ability of replenishing a lubricant as with a coating-type magnetic recording medium.

In the case where an adhesion force between a lubricant and a protective film disposed at a surface of a magnetic layer is weak, moreover, a film thickness of the lubricant is reduced during heating or sliding hence accelerating abrasion. Therefore, a large amount of the lubricant is required. The large amount of the lubricant is the mobile lubricant, and therefore a function of replenishing the lost lubricant can be provided. However, an excessive amount of the lubricant makes the film thickness of the lubricant larger than the surface roughness. Therefore, a problem associated with adhesion arises, and in a crucial case, sticktion arises to cause driving failures. The above-described problems associated with the friction have not been sufficiently solved by conventional perfluoropolyether (PFPE)-based lubricants.

Particularly for a thin film magnetic recording medium having high surface smoothness, a novel lubricant is designed at a molecular level, and synthesized to solve the above-described trade-off. Moreover, there are a number of reports regarding lubricity of PFPE. As described, lubricants are very important in magnetic recording media.

Chemical structures of typical PFPE-based lubricants are depicted in Table 1.

TABLE 1
Fomblin-based lubricants

| | |
|---|---|
| | X—CF₂(OCF₂CF₂)ₙ(OCF₂)ₘOCF₂—X(0.5 < n/m < 1) |
| Z | X = —OCF₃ |
| Z-DOL | X = —CH₂OH |
| Z-DIAC | X = —COOH |
| Z-Tetraol | X = —CH₂ OCH₂OHOH₂ OH |
| | OH |
| AM2001 | 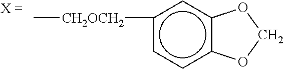 |

Other lubricants

| | |
|---|---|
| A20H | 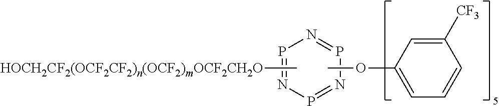 |
| Mono | F—(CF₂CF₂CF₂O)₁—CF₂CF₂CH₂—N(C₃H₇)₂ |

Z-DOL in Table 1 is one of lubricants typically used for thin-film magnetic recording media. Moreover, Z-tetraol is a lubricant, in which a functional hydroxyl group is further introduced into a main chain of PFPE, and it has been reported that use of Z-tetraol enhances reliability of a drive while reducing a space at an interface between a head and a medium. It has been reported that A20H suppresses decomposition of the PFPE main chain with Lewis acid or Lewis base, and improves tribological properties. On the other hand, it has been reported that Mono has a different polymer main chain and different polar groups to those of the PFPE, and the polymer main chain and polar groups of Mono are respectively poly-n-propyloxy, and amine, and Mono reduces adhesion interactions at near contact.

However, a typical solid lubricant, which has a high melting point and is considered thermally stable, disturbs an electromagnetic conversion process that is extremely highly sensitive, and moreover, an abrasion powder scraped by a head is generated on a running track. Therefore, abrasion properties are deteriorated. As described above, the liquid lubricant has mobility that enables to move the adjacent lubricant layer to replenish the lubricant removed due to abrasion by the head. However, the lubricant is span-off from a surface of the disk especially at a high temperature during driving of the disk, because of the mobility of the lubricant, and thus the lubricant is reduced. As a result, a protection function is lost. Accordingly, a lubricant having a high viscosity and low volatility is suitably used, and use of such a lubricant enables to prolong a service life of a disk drive with suppressing an evaporation rate.

Considering the above-described lubricating systems, requirements for a low-friction and low-abrasion lubricant used for thin film magnetic recording media are as follows.

(1) Low volatility.
(2) Low surface tension for a surface filling function.
(3) Interaction between terminal polar groups and a surface of a disk.
(4) High thermal and oxidization stability in order to avoid decomposition or reduction over a service period.
(5) Chemically inactive with metals, glass, and polymers, and no abrasion powder generated from a head or a guide.
(6) No toxicity and no flammability.
(7) Excellent boundary lubricating properties.
(8) Soluble with organic solvents.

Recently, an ionic liquid has been attracted attentions as one of solvents for synthesis of organic or inorganic materials and being friendly to the environments in the fields of electricity storage materials, a separation technology; and a catalyst technology. The ionic liquid is roughly classified as a molten salt having a low melting point. The ionic liquid is typically a molten salt having a melting point of 100° C. or lower, among the above-mentioned molten salts. The important properties of the ionic liquid used as a lubricant are low volatility, inflammability, thermal stability, and an excellent dissolving performance.

For example, abrasion and wear of a surface of a metal or ceramic may be reduced by using a certain ionic liquid compared to a conventional hydrocarbon-based lubricant. For example, there is a report that, in the case where an imidazole cation-based ionic liquid is synthesized by substituting with a fluoroalkyl group, and tetrafluoroboric acid salt or hexafluorophosphoric acid salt of alkyl imidazolium is used for steel, aluminium, copper, single crystal SiO₂, silicon, or sialon ceramics (Si—Al—O—N), tribological properties more excellent than those of cyclic phosphazene (X-1P) or PFPE are exhibited. Moreover, there is a report that an ammonium-based ionic liquid reduces frictions more than a base oil in the region of elastohydrodynamic to boundary lubrication. Moreover, effects of the ionic liquid as an additive for a base oil have been studied, and a chemical or tribochemical reaction of the ionic liquid has been researched to understand lubricating systems. However, there are almost no application examples of the ionic liquid to magnetic recording media.

Among the ionic liquids, a perfluorooctanoic acid alkyl ammonium salt is a protic ionic liquid (PIL), and has been reported as having a significant effect of reducing frictions of magnetic recording media compared to Z-DOL mentioned above (see, for example, PTLs 1 and 2, and NPLs 1 to 3).

However, the above-mentioned perfluorocarboxylic acid ammonium salts have weak interaction between a cation and an anion in the reaction represented by the following reaction formula (A). According to Le Chatelier's principle, the equilibrium of the reaction is sifted to the left side at a high temperature, and the perfluorocarboxylic acid ammonium salt becomes a dissociated neutral compound and hence thermal stability is deteriorated. Specifically, protons are transferred at a high temperature, the equilibrium is sifted to neutral substance and dissociation occurs (see, for example, NPL 4). Moreover, it has been pointed out that friction properties are poor at high temperatures (see NPL 6).

CₙF₂ₙ₊₁COOH+CₙF₂ₙ₊₁NH₂⇄CₙF₂ₙ₊₁COO⁻H₃N⁺CₙH₂ₙ₊₁ (A)

Watanabe et al. have reported that proton mobility and thermal stability of a protic ionic liquid largely depend on an acid dissociation constant difference ΔpKa between an acid and a base, and the thermal stability of the ionic liquid is significantly improved by using an acid that makes ΔpKa or greater, when DBU (1,8-diazabicyclo[5,4,0]undec-7-ene) is used as the base (see NPL 5).

Kondo et al. have proposed that an octadecyl ammonium perfluorooctanoate-based protic ionic liquid having a large value of ΔpKa improves durability of a magnetic recording medium (see NPLs 6 to 8 and PTL 3).

Meanwhile, the limit of a surface recording density of a hard disk is said to be from 1 Tb/in² to 2.5 Tb/in². Currently, the surface recording density is getting close to the limit, but developments of technology for increasing capacities have been actively conducted with a reduction in particle size of magnetic particles as a premise. As the technology for increasing capacities, there are a reduction in an effective flying height and introduction of Shingle Write (BMP).

Moreover, there is “thermally-assisted magnetic recording (heat assisted magnetic recording)” as the next-generation recording technology. The thermally-assisted magnetic recording is schematically illustrated in FIG. 1. In FIG. 1, the referential numeral 1 is laser light, the referential numeral 2 is near field light, the referential numeral 3 is a recording head (PMR element), and the referential numeral 4 is a reproducing head (TMR element). The problems of the thermally-assisted magnetic recording include a deterioration of durability due to evaporation or deterioration of a lubricant on a surface of a magnetic layer because a recording area is heated by laser during recording and reproducing. Even though it is a short period, there is a possibility that a thin film magnetic recording medium is exposed to a high temperature, which is 400° C. or higher, in thermally-assisted magnetic recording. Therefore, thermal stability of a lubricant generally used for thin film magnetic recording media, such as Z-DOL and Z-TETRAOL, is considered.

It is reported as a method of molecule design of a novel ionic liquid with improved thermal stability that a pyrrolidinium-based ionic liquid including germinal dication may improve thermal resistance more than a typical ionic liquid of monocation (see NPL 9). As described in NPL 9, however, a relationship between a molecular structure constituting the pyrrolidinium-based ionic liquid and physical or chemical characteristics has not been fully understood yet. A combination of a cation and an anion largely influences on physical or chemical characteristics of an ionic liquid. A variety of the anion site is many, but the relationship could not be clarified unless the cation is a cation structurally similar to the anion (see, for example, NPL 10). For example, viscosity of the liquid increases, as hydrogen bonding strength of halogen is stronger (Cl>Br>I). However, the method for increasing the viscosity is not limited to the increase in the hydrogen bonding strength. For example, the viscosity can be increased by varying an alkyl chain of imidazole. Similarly, the combination of the anion and cation influences on a melting point, surface tension, and thermal stability, but a wide range of researches has not be conducted on an effect of the anion. Accordingly, it is possible to change physical or chemical characteristics of an ionic liquid by with a combination of cations or anions, but it is difficult to predict.

There are numbers of reported examples of a dicationic ionic liquid, but there are not so many reports related to a dianionic ionic liquid (see NPLs 11 to 13). In most of the above-mentioned reports, the application of the ionic liquid is electrochemical use or use as an organic synthesis solvent, or the application is not specifically disclosed. Moreover, in the above-mentioned reports, a strong acid, i.e., perfluorosulfonic acid or perfluorosulfonylimide having a small value of pKa has not been reported.

### CITATION LIST

### Patent Literatures

PTL 1: Japanese Patent No. 2581090
PTL 2: Japanese Patent No. 2629725
PTL 3: International Publication No. WO 2014/104342

### Non-Patent Literatures

NPL 1: Kondo, H., Seto, J., Haga. S., Ozawa, K., (1989) Novel Lubricants for Magnetic Thin Film Media, Magnetic Soc. Japan, Vol. 13, Suppl. No. S1, pp. 213-218
NPL 2: Kondo, H., Seki, A., Watanabe, H., & Seto, J., (1990). Frictional Properties of Novel Lubricants for Magnetic Thin Film Media, IEEE Trans. Magn. Vol. 26, No. 5, (September 1990), pp. 2691-2693, ISSN:0018-9464
NPL 3: Kondo, H., Seki, A., & Kita, A., (1994a). Comparison of an Amide and Amine Salt as Friction Modifiers for a Magnetic Thin Film Medium. Tribology Trans. Vol. 37, No. 1, (January 1994), pp. 99-105, ISSN: 0569-8197
NPL 4: Yoshizawa, M., Xu, W., Angell, C. A., Ionic Liquids by Proton Transfer: Vapor pressure, Conductivity, and the Relevance of ΔpKa from Aqueous Solutions, J. Am. Chem. Soc., Vol. 125, pp. 15411-15419 (2003)
NPL 5: Miran, M. S., Kinoshita, H., Yasuda, T., Susan, M. A. B. H., Watanabe, M., Physicochemical Properties Determined by ΔpKa for Protic Ionic Liquids Based on an Organic Super-strong Base with Various Bronsted Acids, Phys. Chem. Chem. Phys., Vol 14, pp. 5178-5186 (2012)
NPL 6: Hirofumi Kondo, Makiya Ito, Koki Hatsuda, KyungSung Yun and Masayoshi Watanabe, “Novel Ionic Lubricants for Magnetic Thin Film” Media, IEEE TRANSACTIONS ON MAGNETICS, VOL. 49, NO. 7, pp. 3756-3759, JULY (2013)
NPL 7: Hirofumi Kondo, Makiya Ito, Koki Hatsuda, Nobuo Tano, KyungSung Yun and Masayoshi Watanabe, IEEE International magnetic conference Dresden, Germany, May 4-8, 2014
NPL 8: Hirofumi Kondo, Makiya Ito, Koki Hatsuda, Nobuo Tano, KyungSung Yun and Masayoshi Watanabe, IEEE Trans. Magn., 2014, Vol. 50, Issue 11, Article#: 3302504
NPL 9: Anderson, J. L., Ding R., Ellern A., Armstrong D. W., “Structure and Properties of High Stability Geminal DicationicIonic Liquids”, J. Am. Chem. Soc., 2005, 127, 593-604.
NPL 10: Dzyuba, S. V. Bartsch, R. A., “Influence of Structural Variations in 1-Alkyl(aralkyl)-3-Methylimidazolium Hexafluorophosphates and Bis(trifluoromethylsulfonyl)imides on Physical Properties of the Ionic Liquids, Chem. Phys. Phys. Chem. 2002, 3, 161-166
NPL 11: Yukihiro Yoshida, Hirofusa Tanaka, Gunzi Saito, Lahcene Ouahab, Hiroyuki Yoshida and Naoki Sato, Inorg. Chem., 2009, 48 (21), pp 9989-9991
NPL 12: Wu Xu and C. Austen Angell, Science 17 Oct. 2003: Vol. 302 no. 5644 pp. 422-425
NPL 13: Jean-Hubert Olivier, Franck Camerel, Gilles Ulrich, Joaquin Barbera and Raymond Ziessel, Chemistry-A European Journal Volume 16, Issue 24, pages 7134-7142, 2010

### SUMMARY OF INVENTION

### Technical Problem

In the field of magnetic recording media, however, problems still remain in practical properties, such as running performances, abrasion resistance, and durability, due to lack of lubricity.

The present invention has proposed based on the above-described situations in the art, and the present invention provides an ionic liquid having excellent lubricity at a high temperature, a lubricant having excellent lubricity at a high temperature, and a magnetic recording medium having excellent practical properties.

### Solution to Problem

The present inventors have diligently conducted researches. As a result, the present inventors have found that an ionic liquid including a conjugate base having 2 or more anions in a molecule of the conjugate base can improve thermal stability and introduction of a long-chain alkyl group reduces a friction coefficient to significantly improve durability. The present invention has been accomplished based on the above-described insight.

<1> A lubricant including:

an ionic liquid including a conjugate acid and a conjugate base having 2 or more anions in a molecule of the conjugate base,

wherein the conjugate acid has a group including a straight-chain hydrocarbon group having 6 or more carbon atoms, and
a pKa of an acid that is a source of the conjugate base in acetonitrile is 10 or less.
<2> The lubricant according to <1>,
wherein the ionic liquid is represented by General Formula (1) below,

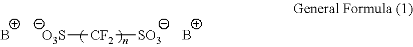

where, in General Formula (1), B⁺ is the conjugate acid and n is 1 or greater but 15 or less.
<3> The lubricant according to <1> or <2>,
wherein the conjugate acid is represented by General Formula (A) below,

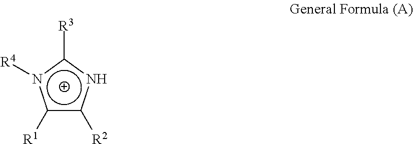

where, in General Formula (A), R¹ and R² are each a hydrogen atom or R¹ and R² may form a benzene ring together with carbon atoms to which R¹ and R² are bonded; R³ is a group including a straight-chain hydrocarbon group having 6 or more carbon atoms; and R⁴ is a hydrogen atom or a hydrocarbon group.
<4> The lubricant according to <1> or <2>,
wherein the conjugate acid is represented by General Formula (B),

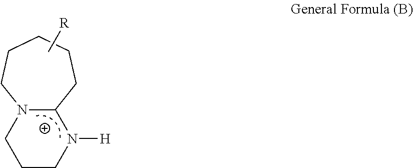

where, in General Formula (B), R is a group bonded to a bicyclic ring and including a straight-chain hydrocarbon group having 6 or more carbon atoms.
<5> A magnetic recording medium including:

a non-magnetic support;

a magnetic layer disposed on the non-magnetic support; and

the lubricant according to any one of <1> to <4>, disposed on the magnetic layer.

<6> An ionic liquid including:

a conjugate acid; and

a conjugate base having 2 or more anions in a molecule of the conjugate base,

wherein the conjugate acid has a group including a straight-chain hydrocarbon group having 6 or more carbon atoms, and
a pKa of an acid that is a source of the conjugate base in acetonitrile is 10 or less.
<7> The ionic liquid according to <6>,
wherein the ionic liquid is represented by General Formula (1) below,

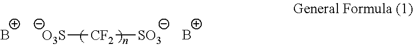

where, in General Formula (1), B⁺ is the conjugate acid and n is 1 or greater but 15 or less.

<8> The ionic liquid according to <6> or <7>,

wherein the conjugate acid is represented by General Formula (A) below,

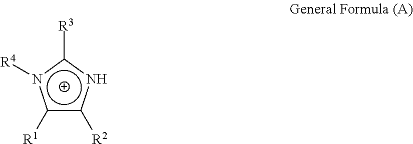

where, in General Formula (A), R¹ and R² are each a hydrogen atom or R¹ and R² may form a ring together with carbon atoms to which R¹ and R² are bonded; R³ is a group including a straight-chain hydrocarbon group having 6 or more carbon atoms; and R⁴ is a hydrogen atom or a hydrocarbon group.
<9> The ionic liquid according to <6> or <7>,
wherein the conjugate acid is represented by General Formula (B),

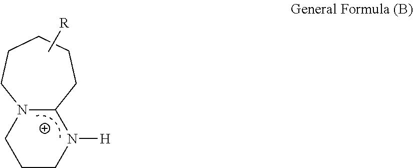

where, in General Formula (B), R is a group bonded to a bicyclic ring and including a straight-chain hydrocarbon group having 6 or more carbon atoms.

### Advantageous Effects of Invention

The present invention can improve thermal stability of a lubricant, such as evaporation and thermal decomposition, to maintain excellent lubricity over a long period. Moreover, the present invention can give excellent lubricity and improve practical properties, such as running performances, abrasion resistance, and durability, when the lubricant is used for a magnetic recording medium.

### BRIEF DESCRIPTION OF DRAWINGS

FIG. 1 is a schematic view illustrating thermally-assisted magnetic recording.

FIG. 2 is a cross-sectional view illustrating one example of a hard disk according to one aspect of the present invention.

FIG. 3 is a cross-sectional view illustrating one example of a magnetic tape according to one aspect of the present invention.

FIG. 4 is a schematic view illustrating a pin-on-disk tester.

FIG. 5 depicts the friction test results.

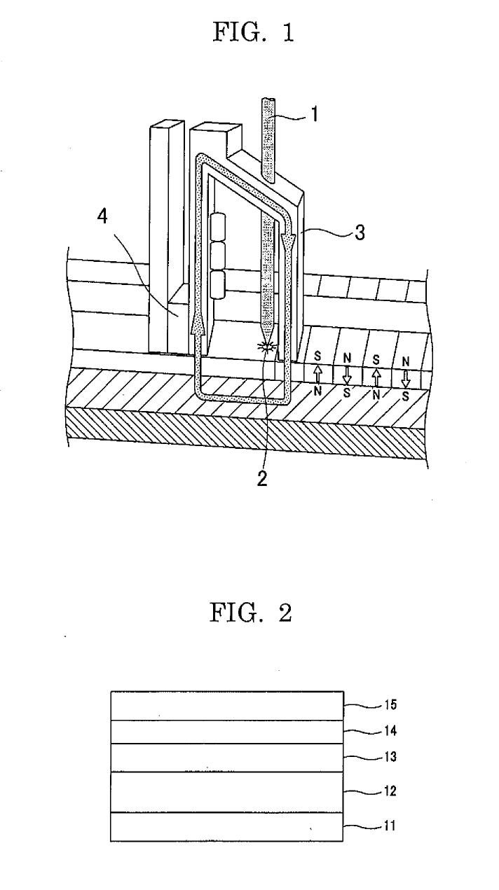

### DESCRIPTION OF EMBODIMENTS

Embodiments of the present invention are specifically described with reference to drawing hereinafter in the following order.

1. Lubricant and ionic liquid
2. Magnetic recording medium

### 3. Examples

### 1. LUBRICANT AND IONIC LIQUID

The lubricant, which is one embodiment of the present invention, includes an ionic liquid including a conjugate acid and a conjugate base having 2 or more anions in a molecule of the conjugate base.

The conjugate base, which is one embodiment of the present invention, includes a conjugate acid and a conjugate base having 2 or more anions in a molecule of the conjugate base.

In the ionic liquid, the conjugate acid has a group including a hydrocarbon group. The hydrocarbon group is a straight-chain hydrocarbon group having 6 or more carbon atoms.

In the ionic liquid, a pKa of an acid that is a source of the conjugate base in acetonitrile is 10 or less.

Since the ionic liquid of the present embodiment includes a conjugate acid a conjugate base having 2 or more anions in a molecule of the conjugate base, and a pKa of an acid that is a source of the conjugate base in acetonitrile is 10 or less, excellent thermal stability can be exhibited. Since the cation site has a group including a hydrocarbon group having 6 or more carbon atoms, moreover, excellent lubricity can be obtained in combination with the thermal stability.

In the present specification, “pKa” is an acid dissociation constant, and is an acid dissociation constant in acetonitrile.

The pKa is 10 or less, which is a strong acid, and is preferably 6.0 or less. The lower limit of the pKa is not particularly limited and may be appropriately selected depending on the intended purpose, but the pKa is preferably −5.0 or greater.

### <<Conjugate Base>>

The conjugate base has 2 or more anions in a molecule of the conjugate base, and preferably has 2 anions in a molecule of the conjugate base.

Examples of the conjugate base include conjugate bases represented by General Formula (X) below.

^{⊖}ZYZ^{⊖} General Formula (X)

In General Formula (X), Z⁻ and Z′⁻ are each independently an anionic functional group; and Y is a linking group bonded to Z⁻ and Z′⁻ with covalent bonds to link Z⁻ and Z′⁻.

Examples of the anionic functional group include anionic functional groups represented by structural formulae and a general formula below.

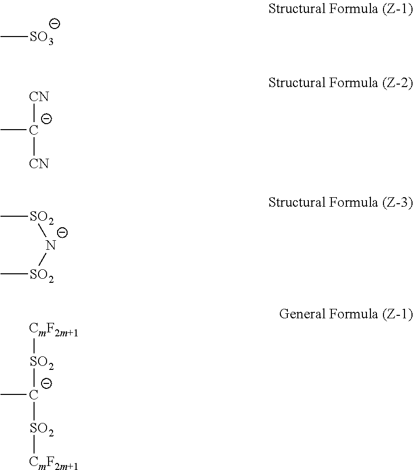

In General Formula (Z-1), m is 1 or greater but 10 or less, and is preferably 1 or greater but 6 or less.

Note that, a bond in each structural formula and the general formula is bonded to Y.

Y is preferably a fully fluorinated hydrocarbon group. Examples of the fully fluorinated substituted include groups represented by General Formula (Y-1) below.

CF₂ₙ General Formula (Y-1)

In General Formula (Y-1), n is 1 or greater but 15 or less, and preferably 1 or greater but 10 or less.

Examples of the conjugate base include conjugate bases represented by General Formula (X-1) below, conjugate bases represented by General Formula (X-2) below, conjugate bases represented by General Formula (X-3) below, conjugate bases represented by General Formula (X-4) below, and conjugate bases represented by General Formula (X-5) below.

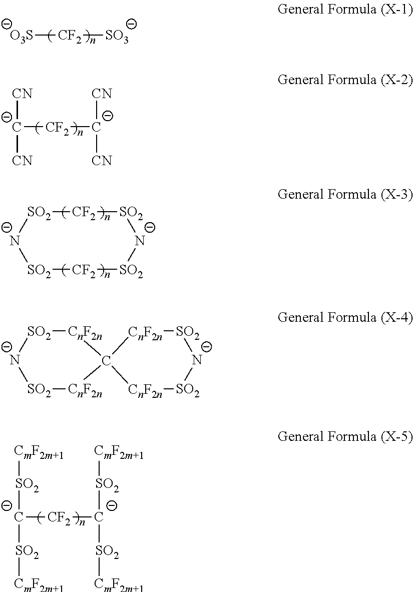

In General Formulae (X-1) to (X-5), n is each independently 1 or greater but 15 or less, preferably 1 or greater but 10 or less; and m is each independently 1 or greater but 10 or less, preferably 1 or greater but 6 or less.

In General Formula (X-4), —CₙF₂ₙ— is preferably —(CF₂)ₙ—.

### <<Conjugate Acid>>

The conjugate acid has a group including a straight-chain hydrocarbon group having 6 or more carbon atoms.

The upper limit of the number of carbon atoms in the straight-chain hydrocarbon group having 6 or greater carbon atoms is not particularly limited and may be appropriately selected depending on the intended purpose. The upper limit of the number of carbon atoms is preferably 30 or less, more preferably 25 or less, and particularly preferably 20 or less in view of availability of raw materials. When the hydrocarbon group has a long chain, lubricity can be improved, with reducing a coefficient of friction.

The group including a straight-chain hydrocarbon group having 6 or more carbon atoms is preferably a straight-chain hydrocarbon group having 6 or more carbon atoms.

The hydrocarbon group may be a saturated hydrocarbon group, or an unsaturated hydrocarbon group partially including a double bond, or an unsaturated branched hydrocarbon group partially including a branched structure, as long as the hydrocarbon group is a straight-chain hydrocarbon group. Among the above-listed examples, an alkyl group, which is a saturated hydrocarbon group, is preferably in view of abrasion resistance. Moreover, the hydrocarbon group is preferably a straight-chain hydrocarbon group that does not include a branched structure even at part.

The conjugate acid is not particularly limited and may be appropriately selected depending on the intended purpose. The conjugate acid is preferably conjugate acids represented by General Formula (A) below and conjugate acids represented by General Formula (B) below.

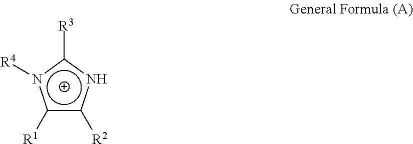

In General Formula (A), R¹ and R² are each a hydrogen atom or R¹ and R² may form a ring (e.g. a benzene ring) together with carbon atoms to which R¹ and R² are bonded; R³ is a group including a straight-chain hydrocarbon group having 6 or more carbon atoms; and R⁴ is a hydrogen atom or a hydrocarbon group.

Note that, the conjugate acid represented by General Formula (A) may have another resonance structure (intrinsic structure). Specifically, the conjugate acid represented by General Formula (A) may have a resonance structure (intrinsic structure) where a nitrogen atom to which R⁴ is bonded is positively charged and a hydrogen atom is bonded to the nitrogen atom. In the present invention, the conjugate acid having the above-described resonance structure (intrinsic structure) is also included in the conjugate acid represented by General Formula (A). It is the same in General Formula (A-1), General Formula (A-2), and General Formula (2) below.

The upper limit of the number of carbon atoms of the straight-chain hydrocarbon group having 6 or more carbon atoms for R³ is not particularly limited and may be appropriately selected depending on the intended purpose. In view of readily availability of raw materials, the upper limit is preferably 30 or less, more preferably 25 or less, and particularly preferably 20 or less. Since the hydrocarbon group is a long chain, a friction coefficient can be reduced and lubricity can be improved.

R³ is preferably the straight-chain hydrocarbon group having 6 or more carbon atoms.

The hydrocarbon group of R³ is not limited as long as the hydrocarbon group is a straight chain. The hydrocarbon group of R³ may be a saturated hydrocarbon group, an unsaturated hydrocarbon group having a double bond at part, or an unsaturated branched hydrocarbon group having a branched chain at part. Among the above-listed examples, the hydrocarbon group is preferably an alkyl group that is a saturated hydrocarbon group in view of abrasion resistance. Moreover, the hydrocarbon group is also preferably a straight-chain hydrocarbon group that does not a branched chain at any part.

The hydrocarbon group of R⁴ is not particularly limited and may be appropriately selected depending on the intended purpose. The hydrocarbon group of R⁴ is preferably a straight-chain hydrocarbon group having 6 or more carbon atoms. The straight-chain hydrocarbon group having 6 or more carbon atoms is preferably the hydrocarbon group described related to R³.

The conjugated acid represented by General Formula (A) is preferably any of conjugated acids represented by General Formula (A-1) and General Formula (A-2) below.

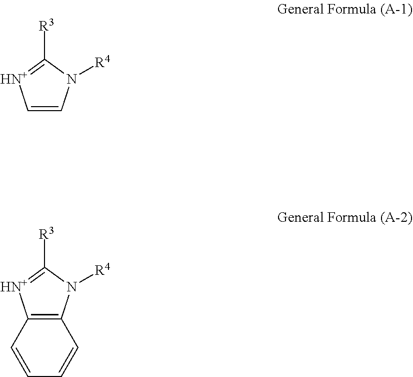

In General Formula (A-1) and General Formula (A-2), R³ is a group including a straight-chain hydrocarbon group having 6 or more carbon atoms; and R⁴ is a hydrogen atom or a hydrocarbon group. Examples of R³ and R⁴ are identical to the examples of R³ and R⁴ in General Formula (A).

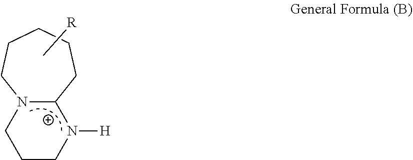

In General Formula (B), R is a group bonded to a bicyclic ring and including a straight-chain hydrocarbon group having 6 or more carbon atoms.

Note that, the conjugate acid represented by General Formula (B) may have another resonance structure (intrinsic structure). Specifically, the conjugate acid represented by General Formula (B) may have a resonance structure (intrinsic structure) where H is bonded to another nitrogen atom. In the present invention, the conjugate acid having the above-described resonance structure (intrinsic structure) is also included in the conjugate acid represented by General Formula (B). It is the same in General Formula (B-1) and General Formula (3) below.

The upper limit of the number of carbon atoms of the straight-chain hydrocarbon group having 6 or more carbon atoms for R is not particularly limited and may be appropriately selected depending on the intended purpose. In view of readily availability of raw materials, the upper limit is preferably 30 or less, more preferably 25 or less, and particularly preferably 20 or less. Since the hydrocarbon group is a long chain, a friction coefficient can be reduced and lubricity can be improved.

R is preferably the straight-chain hydrocarbon group having 6 or more carbon atoms.

The hydrocarbon group of R is not limited as long as the hydrocarbon group is a straight chain. The hydrocarbon group of R may be a saturated hydrocarbon group, an unsaturated hydrocarbon group having a double bond at part, or an unsaturated branched hydrocarbon group having a branched chain at part. Among the above-listed examples, the hydrocarbon group is preferably an alkyl group that is a saturated hydrocarbon group in view of abrasion resistance. Moreover, the hydrocarbon group is also preferably a straight-chain hydrocarbon group that does not a branched chain at any part.

The conjugate acid represented by General Formula (B) is preferably a conjugate acid represented by General Formula (B-1) below.

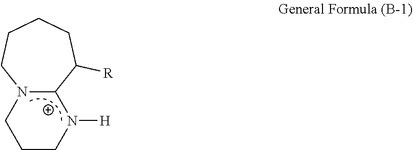

In general formula (B-1), R is a group including a straight-chain hydrocarbon group having 6 or more carbon atoms. Examples of R are identical to the examples of R in General Formula (B).

### <<Preferable Examples of Ionic Liquid>>

The ionic liquid is preferably an ionic liquid represented by General Formula (1) below, more preferably an ionic liquid represented by General Formula (2) below or an ionic liquid represented by General Formula (3) below.

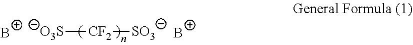

In General Formula (1), B⁺ is the conjugate acid and n is 1 or greater but 15 or less.

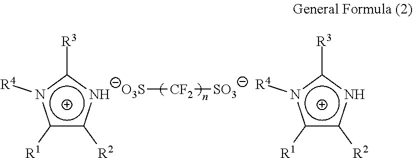

In General Formula (2), R¹ and R² are each a hydrogen atom or R¹ and R² may form a benzene ring together with carbon atoms to which R¹ and R² are bonded; R³ is a group including a straight-chain hydrocarbon group having 6 or more carbon atoms; R⁴ is a hydrogen atom or a hydrocarbon group; and n is 1 or greater but 15 or less. Note that, two of R¹, two of R², two of R³, and two of R⁴ may be the same or different.

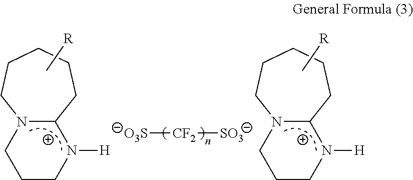

In General Formula (3), R is each independently a group bonded to a bicyclic ring and including a straight-chain hydrocarbon group having 6 or more carbon atoms; and n is 1 or greater but 15 or less.

Examples of the substituents, examples of the repeating units, and preferable ranges of the repeating units in General Formulae (1) to (3) are identical to the examples and preferable ranges of the above-described conjugate acid and conjugate base.

A synthesis method of the ionic liquid is not particularly limited and may be appropriately selected depending on the intended purpose. For example, various types of the ionic liquid can be synthesized with reference to the method disclosed in Examples below.

The ionic liquid of the present embodiment may be used alone as the lubricant, or the ionic liquid may be used in combination with a conventional lubricant.

Examples of the lubricant used in combination include long-chain carboxylic acid, long-chain carboxylic acid ester, perfluoroalkyl carboxylic acid ester, perfluoroalkyl carboxylate, perfluoroalkyl perfluoroalkylcarboxylate, and a perfluoropolyether derivative.

Moreover, an extreme pressure agent may be used in combination at a mass ratio of about 30:70 to about 70:30 in a mass ratio in order to maintain a lubricating effect under severe conditions. The extreme pressure agent reacts with a surface of a metal with friction heat generated when the lubricant is partially in contact with the metal in a boundary lubrication region, and forms a coating film of a reaction product. As a result, friction and abrasion are prevented. As the extreme pressure agent, for example, any of a phosphorus-based extreme pressure agent, a sulfur-based extreme pressure agent, a halogen-based extreme pressure agent, an organic metal-based extreme pressure agent, or a complex extreme pressure agent can be used.

Moreover, an anti-rust agent may be optionally used in combination. The anti-rust agent may be any anti-rust agent typically used for this kind of magnetic recording media. Examples of the anti-rust agent include phenols, naphthols, quinones, heterocyclic compounds containing a nitrogen atom, heterocyclic compounds containing an oxygen atom, and heterocyclic compounds containing a sulfur atom. Moreover, the anti-rust agent may be mixed with the lubricant. Alternatively, the anti-rust agent and the lubricant may be deposited as two or more layers by forming a magnetic layer on a non-magnetic support, and applying an anti-rust agent layer on the upper part of the magnetic layer, followed by applying a lubricant layer.

As a solvent of the lubricant, for example, a single use or a combination of alcoholic solvents, such as isopropyl alcohol (IPA), and ethanol, can be used. For example, a mixture of a hydrocarbon-based solvent, such as normal-hexane, and a fluorine-based solvent can be used.

### 2. MAGNETIC RECORDING MEDIUM

Next, a magnetic recording medium using the above-described lubricant is described. A magnetic recording medium described as one embodiment of the present invention includes at least a magnetic layer on a non-magnetic support, and the above-described lubricant is held on the magnetic layer.

The lubricant of the present embodiment can be applied for so-called a thin film-metal-type magnetic recording medium, in which a magnetic layer formed on a non-magnetic support by a method, such as vapor deposition and sputtering. Moreover, the lubricant can be also applied for a magnetic recording medium having a structure, in which a base layer is disposed between a non-magnetic support and a magnetic layer. Examples of such a magnetic recording medium include a magnetic disk, and a magnetic tape.

FIG. 2 is a cross-sectional view illustrating one example of a hard disk. The hard disk has a structure, in which a substrate 11, a base layer 12, a magnetic layer 13, a protective carbon layer 14, and a lubricant layer 15 are sequentially laminated.

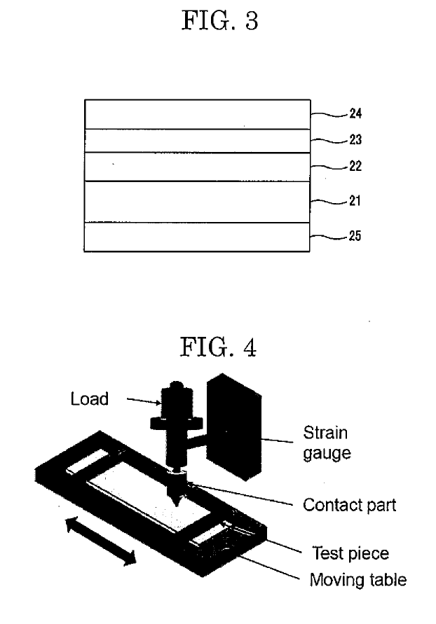

Moreover, FIG. 3 is a cross-sectional view illustrating one example of a magnetic tape. The magnetic tape has a structure, in which a back-coating layer 25, a substrate 21, a magnetic layer 22, a protective carbon layer 23, and a lubricant layer 24 are sequentially laminated.

In the magnetic disk illustrated in FIG. 2, each of the substrate 11 and the base layer 12 corresponds to the non-magnetic support. In the magnetic tape illustrated in FIG. 3, the substrate 21 corresponds to the non-magnetic support. In the case where a rigid substrate, such as an Al alloy plate, and a glass plate, is used as the non-magnetic support, a surface of the substrate may be made hard by forming an oxidized film, such as anodizing or a Ni—P coating on the surface of the substrate.

Each of the magnetic layers 13 and 22 is formed as a continuous film by a method, such as plating, sputtering, vacuum deposition, and plasma CVD. Examples of the magnetic layers 13 and 22 include: longitudinal magnetic recording metal magnetic films formed of metals (e.g., Fe, Co, and Ni), Co—Ni-based alloys, Co—Pt-based alloys, Co—Ni—Pt-based alloys, Fe—Co-based alloys, Fe—Ni-based alloys, Fe—Co—Ni-based alloys, Fe—Ni—B-based alloys, Fe—Co—B-based alloys, or Fe—Co—Ni—B-based alloys; and perpendicular magnetic recording metal magnetic thin films, such as Co—Cr-based alloy thin films, and Co—O-based thin films.

In the case where a longitudinal magnetic recording metal magnetic thin film is formed, particularly, a non-magnetic material, such as Bi, Sb, Pb, Sn, Ga, In, Ge, Si, and Tl, is formed as a base layer 12 on a non-magnetic support in advance, and a metal magnetic material is deposited through vapor deposition or sputtering in a perpendicular direction to diffuse the non-magnetic material into the magnetic metal thin film, to thereby improve a coercive force as well as eliminating orientation to assure in-plane isotropy.

Moreover, a hard protective layer 14 or 23, such as a carbon film, a diamond-formed carbon film, a chromium oxide film, and SiO₂ film, may be formed on a surface of the magnetic layer 13 or 22.

Examples of a method for applying the above-mentioned lubricant to such a metal thin film magnetic recording medium include a method for top-coating a surface of the magnetic layer 13 or 22, or a surface of the protective layer 14 or 23 with the lubricant, as illustrated in FIGS. 2 and 3. A coating amount of the lubricant is preferably from 0.1 mg/m² to 100 mg/m², more preferably from 0.5 mg/m² to 30 mg/m², and particularly preferably from 0.5 mg/m² to 20 mg/m².

As illustrated in FIG. 3, moreover, a metal thin film magnetic tape may optionally have a back-coating layer 25, other than a metal magnetic thin film, which is the magnetic layer 22.

The back-coating layer 25 is formed by adding a carbon-based powder for imparting conductivity, or an inorganic pigment for controlling a surface roughness to a resin binder, and applying the resin binder mixture. In the present embodiment, the above-described lubricant may be internally added to the back-coating layer 25, or applied to the back-coating layer 25 as top coating. Moreover, the above-described lubricant may be internally added to both the magnetic layer 22 and the back-coating layer 25, or applied to both the magnetic layer 22 and the back-coating layer 25 as top coating.

As another embodiment, moreover, the lubricant can be applied for a so-called coating-type magnetic recording medium, in which a magnetic coating film is formed as a magnetic layer by applying a magnetic coating material onto a surface of a non-magnetic support. In the coating-type magnetic recording medium, the non-magnetic support, a magnetic powder constituting the magnetic coating film, and the resin binder for use can be selected from any of those known in the art.

Examples of the non-magnetic support include: polymer substrates formed of polymer materials, such as polyesters, polyolefins, cellulose derivatives, vinyl-based resins, polyimides, polyamides, and polycarbonate; metal substrates formed of aluminium alloys, or titanium alloys; ceramic substrates formed of alumina glass; and glass substrates. Moreover, a shape of the non-magnetic support is not particularly limited, and may be any form, such as a tape, a sheet, and a drum. Furthermore, the non-magnetic support may be subjected to a surface treatment to form fine irregularities in order to control surface properties of the non-magnetic support.

Examples of the magnetic powder include: ferromagnetic iron oxide-based particles, such as γ-Fe₂O₃, cobalt-coated γ-Fe₂O₃; ferromagnetic chromium dioxide; ferromagnetic metal-based particles formed of a metal, such as Fe, Co, and Ni, or an alloy containing any of the above-listed metals; and hexagonal ferrite particles in the form of hexagonal plates.

Examples of the resin binder include: polymers, such as vinyl chloride, vinyl acetate, vinyl alcohol, vinylidene chloride, acrylic acid ester, methacrylic acid ester, styrene, butadiene, and acrylonitrile; copolymers combining two or more selected from the above-listed polymers; polyurethane resins; polyester resins; and epoxy resins. In order to improve dispersibility of the magnetic powder, a hydrophilic polar group, such as a carboxylic acid group, a carboxyl group, and a phosphoric acid group, may be introduced into any of the above-listed binders.

Other than the magnetic powder and the resin binder, additives, such as a dispersing agent, an abrasive, an antistatic agent, and an anti-rust agent, may be added to the magnetic coating film.

As a method for retaining the above-described lubricant in the coating-type magnetic recording medium, there are a method where the lubricant is internally added to the magnetic layer constituting the magnetic coating film formed on the non-magnetic support, a method where the lubricant is applied on a surface of the magnetic layer as top coating, and a combination of the above-listed methods. In the case where the lubricant is internally added into the magnetic coating film, the lubricant is added in an amount of from 0.2 parts by mass to 20 parts by mass relative to 100 parts by mass of the resin binder.

In the case where a surface of the magnetic layer is top-coated with the lubricant, moreover, a coating amount of the lubricant is preferably from 0.1 mg/m² to 100 mg/m², and more preferably from 0.5 mg/m² to 20 mg/m². As a deposition method in the case where the lubricant is applied as top coating, the ionic liquid is dissolved in a solvent, and the obtained solution may be applied or sprayed, or a magnetic recording medium may be dipped in the solution.

The magnetic recording medium, to which the lubricant of the present embodiment is applied, exhibits excellent running performances, abrasion resistance, and durability because of a lubricating effect, and can further improve thermal stability.

### EXAMPLES

### 3. Examples

Specific examples of the present invention are explained below. In the examples, ionic liquids were synthesized, and lubricants including the ionic liquids were produced. Then, magnetic disks and magnetic tapes were produced using the lubricants lubricant, and durability of each of the disks and durability of each of the tapes were evaluated. Production of a magnetic disk, a durability test of the disk, production of a magnetic tape, and a durability test of the tape were performed in the following manner. Note that, the present invention is not limited to these examples.

### <Production of Magnetic Disk>

A magnetic thin film was formed on a glass substrate to produce a magnetic disk as illustrated in FIG. 2, for example, according to International Patent Publication No. WO2005/068589. Specifically, a chemically reinforced glass disk, which was formed of aluminium silicate glass and had an outer diameter of 65 mm, an inner diameter of 20 mm, and a disk thickness of 0.635 mm, was prepared, and a surface of the glass disk was polished so that Rmax of the surface was to be 4.8 nm, and Ra of the surface was to be 0.43 nm. The glass substrate was subjected to ultrasonic cleaning for 5 minutes each in pure water and in isopropyl alcohol (IPA) having the purity of 99.9% or greater, and the washed glass substrate was left to stand in saturated IPA steam for 1.5 minutes, followed by drying. The resultant glass substrate was provided as a substrate 11.

On the substrate 11, a NiAl alloy (Ni: 50 mol %, Al: 50 mol %) thin film in the thickness of 30 nm as a seed layer, a CrMo alloy (Cr: 80 mol %, Mo: 20 mol %) thin film in the thickness of 8 nm as a base layer 12, and a CoCrPtB alloy (Co: 62 mol %, Cr: 20 mol %, Pt: 12 mol %, B: 6 mol %) thin film in the thickness of 15 nm as a magnetic layer 13 were sequentially formed by DC magnetron sputtering.

Subsequently, a 5 nm-thick protective carbon layer 14 formed of amorphous diamond-like carbon was formed by plasma CVD, and the resultant disk sample was subjected to ultrasonic cleaning for 10 minutes in isopropyl alcohol (IPA) having the purity of 99.9% or greater inside a cleaner to remove impurities on a surface of the disk, followed by drying. Thereafter, an IPA solution of an ionic liquid was applied on a surface of the disk by dip coating in the environment of 25° C. and 50% in relative humidity (RH), to form about 1 nm of a lubricant layer 15.

### <Measurement of Thermal Stability>

In the TG/DTA measurement, the measurement was performed by means of EXSTAR6000 available from Seiko Instruments Inc. at a temperature range of from 30° C. to 600° C. at a heating rate of 10° C./min with introducing air at a flow rate of 200 mL/min.

### <Disk Durability Test 1 (Pin-On-Disk Test)>

By means of a pin-on-disk tester illustrated in FIG. 4, friction force caused against the arm was detected by a strain gauge under the following measurement conditions.

### [Measurement Conditions]

Load: 15 g
Sliding speed: 1.7 mm/sec (100 mm/min)
Contact area: 3 mm in diameter, steel ball
Sliding distance: 20 mm
Sliding times: 100 times

### <Disk Durability Test 2>

A CSS durability test was performed by means of a commercially available strain-gauge-type disk friction-abrasion tester in the following manner. A hard disk was mounted on a rotatable spindle with tightening torque of 14.7 Ncm. Thereafter, a head slider was attached on the hard disk in a manner that a center of an air bearing surface at the inner circumference side of the head slider relative to the hard disk was 17.5 mm from a center of the hard disk. The head used for the measurement was an IBM3370-type inline head, a material of the slider was Al₂O₃—TiC, and the head load was 63.7 mN. In the test, the maximum value of friction force was monitored per CSS (contact, start, and stop) in the environment of 100 in cleanliness, 25° C., and 60% RH. The number of times when a coefficient of friction was greater than 1.0 was determined as a result of the CSS durability test. When a result of the CSS durability test was greater than 50,000 times, the result was represented as “>50,000.” Moreover, a CSS durability test was similarly performed after performing a heating test for 3 minutes at a temperature of 300° C. or 350° C., in order to study heat resistance.

### <Production of Magnetic Tape>

A magnetic tape having a cross-sectional structure as illustrated in FIG. 3 was produced. First, Co was deposited on a substrate 21 formed of a 5 μm-thick MICTRON (aromatic polyamide) film available from TORAY INDUSTRIES, INC. by oblique deposition to form a magnetic layer 22 formed of a ferromagnetic metal thin film having a film thickness 100 nm. Next, a protective carbon layer 23 formed of a 10 nm-thick diamond-like carbon was formed on a surface of the ferromagnetic metal thin film by plasma CVD, followed by cutting the resultant into a strip having a width of 6 mm. An ionic liquid dissolved in IPA was applied onto the protective carbon layer 23 in a manner that a film thickness of the ionic liquid solution was about 1 nm. In this manner, a lubricant layer 24 is formed on the magnetic layer to thereby produce a sample tape.

### <Tape Durability Test>

Each sample tape was subjected to a measurement of still durability in an environment having a temperature of −5° C. and in an environment having a temperature of 40° C. and 30% RH, and measurements of a coefficient of friction and shuttle durability in an environment having a temperature of −5° C. and in an environment having a temperature of 40° C. and 90% RH. The still durability was evaluated by a decay time of an output in a paused state decayed by −3 dB. The shuttle resistant was evaluated by the number of shuttles taken until an output was reduced by 3 dB when repeated shuttle run was performed for 2 minutes per time. Moreover, a durability test was similarly performed after performing a heating test for 10 minutes at a temperature of 100° C., in order to study heat resistance.

The ionic liquid of the present embodiment includes a conjugate acid and a conjugate base having 2 or more anions in a molecule of the conjugate base, and a pKa of an acid that is a source of the conjugate base in acetonitrile is 10 or less. Moreover, the cation site preferably has a group including a hydrocarbon group having 6 or more carbon atoms. Thermal stability of such an ionic liquid and durability of a magnetic recording medium using the ionic liquid were studied.

### Example 1A

### Synthesis of 1,3-bis(1-octadecyl-2-heptadecylimidazolium)hexafluoropropanedisulfonate

Synthesis of 1,3-bis(1-octadecyl-2-heptadecylimidazolium)hexafluoropropanedisulfonate was prepared according to the following scheme.

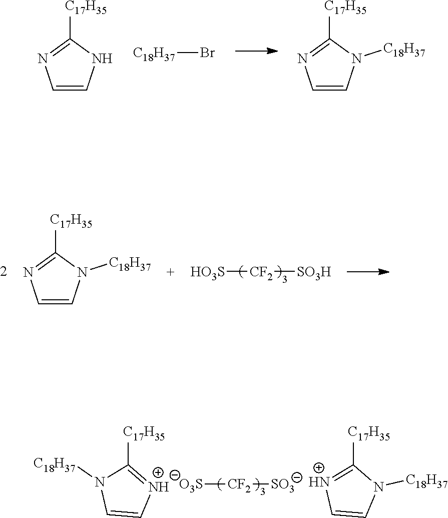

A raw material, 2-heptadecylimidazole, was used after recrystallizing a product obtained from SHIKOKU CHEMICALS CORPORATION with ethanol. Since thermal stability improved by improving the purity from 93% to 98.5% through the recrystallization, the purified product through recrystallization was used as the 2-heptadecyl imidazole used below as a synthesis raw material.

The purified 2-heptadecylimidazole (9.18 g), 9.99 g of octadecyl bromide, and 1.68 g of potassium hydroxide were added to a mixture including 100 mL of acetonitrile and 100 mL of toluene, and the resultant was heated to reflux for 3 hours. The reaction solution was filtered to remove the generated salt, and the solvent was removed by an evaporator. Unreacted raw materials were separated by column chromatography with a solvent of n-hexane/ethyl acetate=9/1 to thereby obtain 14.5 g of 1-octadecyl-2-heptadecylimidazole, which was a target, with the gas chromatography purity of 98% or higher.

In ethanol, 5.94 g of 1-octadecyl-2-heptadecylimidazole was dissolved. To the resultant solution, a solution in which 1.66 g of perfluoropropane-1,3-disulfonic acid was diluted with ethanol was added. After allowing the resultant solution to react at room temperature for 1 hour, the resultant was heated to reflux for 1 hour. After cooling the resultant to room temperature, the ethanol was removed. After sufficiently washing the resultant with water, recrystallization was performed with ethanol to thereby obtain 6.10 g of a product. The yield after recrystallization was 81%.

In the present specification, the measurement of FTIR was performed by means of FT/IR-460 available from JASCO Corporation according to a transmission method using KBr plates or KBr pellets. The resolution of the measurement was 4 cm⁻¹.

The ¹H-NMR and ¹³C-NMR spectra were measured by means of Varian Mercury Plus 300 nuclear magnetic resonance spectrometer (available from Varian, Inc.). A chemical shift of ¹H-NMR was represented with ppm comparing with an internal standard (TMS at 0 ppm or deuterated solvent peak). Splitting patterns were presented by denoting a singlet as s, a doublet as d, a triplet as t, a multiplet as m, and a broad peak as br.

The FTIR absorption of the generated product and the assignment are presented below.

Symmetric stretching vibrations of SO₂ were observed at 1,153 cm⁻¹, symmetric stretching vibrations of CF₂ were observed at 1,269 cm⁻¹, asymmetric stretching vibrations of a SO₂ bond were observed at 1,353 cm⁻¹, bending vibrations of CH₂ were observed at 1,469 cm⁻¹, symmetric stretching vibrations of CH₂ were observed at 2,848 cm⁻¹, asymmetric stretching vibrations of CH₂ were observed at 2,916 cm¹, and stretching vibrations of NH were observed at 3,086 cm⁻¹ and 3,153 cm⁻¹.

Moreover, peaks of a proton (¹H)NMR and a carbon (¹³C)NMR in deuterated chloroform are presented below.

¹H-NMR (CDCl₃, δ ppm); 0.850 (12H, t/J=6.9 Hz), 1.100-1.450 (m, 116H), 1.680-1.830 (m, 8H), 2.896 (4H, t/J=7.5 Hz), 3.985 (4H, t/J=7.5 Hz), 7.135-7.148 (m, 2H), 7.2407-7.264 (m, 2H), 13.379 (brs, 2H)

¹³C-NMR (CDCl₃, δ ppm); 14.085, 22.663, 24.525, 26.388, 27.303, 28.998, 29.349, 29.425, 29.516, 29.608, 29.700, 30.112, 31.898, 47.634, 119.126, 120.881, 147.012

The generated product was determined as 1,3-bis(1-octadecyl-2-heptadecylimidazolium)hexafluoropropanedisulfonate from the spectra above.

Note that, in 1,3-bis(1-octadecyl-2-heptadecylimidazolium)hexafluoropropanedisulfonate, a pKa of an acid [1,3-disulfonic acid perfluoropropane] that is a source of a conjugate base is 0.7 in acetonitrile.

### Example 2A

### Synthesis of 1,3-bis[6-octadecyl-1,8-diazabicyclo[5.4.0]-7-undecenium]hexafluoropropanedisulfonate

Synthesis of 1,3-bis[6-octadecyl-1,8-diazabicyclo[5.4.0]-7-undecenium]hexafluoropropanedisulfonate was performed according to the following scheme.

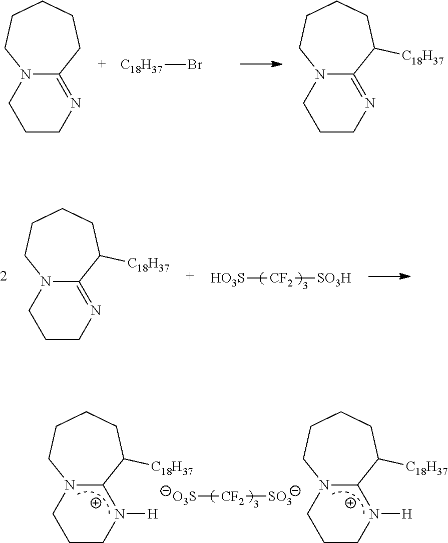

According to the method proposed by Murayama et al. (Non-Patent Literature N. Matsumura, H. Nishiguchi, M. Okada, and S. Yoneda, J. Heterocyclic Chem. pp. 885-887, Vol. 23, Issue 3 (1986)), 6-octadecyl-1,8-diazabicyclo[5.4.0]-7-undecene was synthesized. Specifically, 1,8-diazabicyclo[5.4.0]-7-undecene (DBU) was dissolved in tetrahydrofuran, the resultant solution was cooled to 0° C. To the cooled solution, butyl lithium was added by dripping with stirring for 1 hour at 0° C. To the solution, a THF solution of equimolar octadecyl bromide was added. After completing the reaction, the resultant reaction solution was turned acidic with dilute hydrochloric acid, followed by extraction with diethyl ether. The aqueous solution was turned alkaline with sodium hydroxide, followed by extraction with diethyl ether. The combined diethyl ether layer was washed with water, followed by drying the resultant with anhydrous magnesium sulfate to remove the solvent. Thereafter, purification was performed by silica gel column chromatography to thereby obtain 6-octadecyl-1,8-diazabicyclo[5.4.0]-7-undecene. The yield was 75%.

In ethanol, 3.73 g of 6-octadecyl-1,8-diazabicyclo[5.4.0]-7-undecene was dissolved. To the resultant solution, a solution obtained by diluting 1.83 g of a 78.7% perfluoropropane-1,3-disulfonic acid aqueous solution with ethanol was added, and the mixture was allowed to react for 1 hour at room temperature, followed by heating the resultant to reflux for 1 hour. After cooling the resultant, the solvent was removed, the residue was dissolved in dichloromethane, and the resultant was sufficiently washed with water. Thereafter, the resultant was dried with anhydrous sodium sulfate to remove the solvent, to thereby obtain 4.85 g of 1,3-bis[6-octadecyl-1,8-diazabicyclo[5.4.0]-7-undecenium]hexafluoropropanedisulfonate. The yield was 87%.

The FTIR absorption of the generated product and the assignment are presented below.

Symmetric stretching vibrations of SO₂ were observed at 1,132 cm⁻¹, symmetric stretching vibrations of CF₂ were observed at 1,244 cm⁻¹, bending vibrations of CH₂ were observed at 1,468 cm⁻¹, stretching vibrations of C═N were observed at 1,574 cm⁻¹, symmetric stretching vibrations of CH₂ were observed at 2,850 cm⁻¹, asymmetric stretching vibrations of CH₂ were observed at 2,918 cm⁻¹, and stretching vibrations of NH were observed at 3,288 cm⁻¹ and 3,134 cm⁻¹.

Moreover, peaks of a proton (¹H)NMR and a carbon (¹³C)NMR in deuterated methanol are presented below.

¹H-NMR (deuterated methanol, δ ppm); 0.894 (6H, t/J=7.0 Hz), 1.228-1.400 (m, 64H), 1.499-1.615 (m, 4H), 1.645-1.900 (m, 12H), 1.948-2.098 (m, 4H), 2.818 (m, 2H), 3.362 (4H, t/J=5.7 Hz), 3.510-3.800 (m, 8H)

¹³C-NMR (deuterated methanol, δ ppm); 14.468, 20.351, 23.740, 26.950, 27.067, 28.425, 30.059, 30.486, 30.562, 30.608, 30.715, 30.791, 33.081, 39.567, 44.253, 50.450, 54.617, 169.105

The generated product was determined as 1,3-bis[6-octadecyl-1,8-diazabicyclo[5.4.0]-7-undecenium]hexafluoropropanedisulfonate from the spectra above.

Note that, in 1,3-bis[6-octadecyl-1,8-diazabicyclo[5.4.0]-7-undecenium]hexafluoropropanedisulfonate, a pKa of an acid [perfluoropropane-1,3-disulfonic acid] that is a source of a conjugate base is 0.7 in acetonitrile.

### Comparative Example 1A

### Synthesis of 6-octadecyl-1,8-diazabicyclo[5.4.0]-7-undeceniumheptadecafluorooctanesulfonate

Synthesis of 6-octadecyl-1,8-diazabicyclo[5.4.0]-7-undeceniumheptadecafluorooctanesulfonate was performed according to the following scheme.

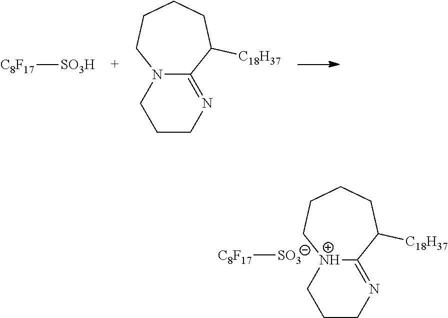

In ethanol, 3.13 g of 6-octadecyl-1,8-diazabicyclo[5.4.0]-7-undecene synthesized in the same manner as in Example 2A was dissolved. To the resultant solution, a solution obtained by diluting 3.87 g of heptadecafluorooctanesulfonic acid with ethanol was added, and the resultant was allowed to react for 1 hour at room temperature. Thereafter, the resultant was heated to reflux for 1 hour. After cooling the resultant, the solvent was removed. The residue was dissolved in a dichloromethane, and the resultant was sufficiently washed with water, followed by drying the resultant with anhydrous sodium sulfate to remove the solvent, to thereby obtain 6.15 g of 6-octadecyl-1,8-diazabicyclo[5.4.0]-7-undeceniumheptadecafluorooctanesulfonate. The yield was 88%.

The FTIR absorption of the generated product and the assignment are presented below.

Symmetric stretching vibrations of CF₂ were observed at adjacent to 1,252 cm⁻¹, bending vibrations of CH₂ were observed at 1,467 cm⁻¹, stretching vibrations of C═N were observed at 1,643 cm⁻¹, symmetric stretching vibrations of CH₂ were observed at 2,851 cm⁻¹, asymmetric stretching vibrations of CH₂ were observed at 2,920 cm⁻¹, and stretching vibrations of NH were observed at the range of 3,178 cm⁻¹ to 3,410 cm⁻¹.

Moreover, peaks of a proton (¹H)NMR and a carbon (¹³C)NMR in deuterated chloroform are presented below.

¹H-NMR (CDCl₃, δ ppm); 0.843 (6H, t/J=7.0 Hz), 1.205-1.287 (m, 32H), 1.544-1.800 (m, 8H), 1.975-2.033 (m, 2H), 2.792-2.816 (m, 1H), 3.440-3.559 (m, 6H), 8.713 (brs, 1H)

¹³C-NMR (CDCl₃, δ ppm); 14.024, 19.336, 22.633, 25.121, 26.311, 27.181, 28.311, 29.028, 29.303, 29.425, 29.532, 29.608, 29.654, 31.882, 38.491, 43.375, 49.725, 53.785, 168.029

The generated product was determined as 6-octadecyl-1,8-diazabicyclo[5.4.0]-7-undeceniumheptadecafluorooctanesulfonate from the spectra above.

Note that, in 6-octadecyl-1,8-diazabicyclo[5.4.0]-7-undeceniumheptadecafluorooctanesulfonate, a pKa of an acid [heptadecafluorooctanesulfonic acid] that is a source of a conjugate base is 0.7 in acetonitrile.

### Example 1B

### <Thermal Stability Measurement Results>

The 5% weight reduction temperature, 10% weight reduction temperature, and 20% weight reduction temperature of 1,3-bis(1-octadecyl-2-heptadecylimidazolium)hexafluoropropanedisulfonate synthesized in Example 1A were 347.7° C., 368.3° C., and 387.9° C., respectively. The endothermic decomposition temperature was 400.5° C. It could be found that the weight reduction temperatures were higher by 150° C. or more compared to the commercially available perfluoropolyether Z-DOL (Comparative Example 2B) that was presented as Comparative Example and was generally known as a lubricant for magnetic recording media, and were higher by 100° C. or more compared to Z-TETRAOL (Comparative Example 3B).

### Example 2B

### <Thermal Stability Measurement Results>

The 5% weight reduction temperature, 10% weight reduction temperature, and 20% weight reduction temperature of 1,3-bis[6-octadecyl-1,8-diazabicyclo[5.4.0]-7-undecenium]hexafluoropropanedisulfonate synthesized in Example 2A were 362.0° C., 383.1° C., and 407.1° C., respectively. The endothermic decomposition temperature was 421.5° C. The 5% weight reduction temperature, 10% weight reduction temperature, 20% weight reduction temperature, and the endothermic decomposition temperature were all higher compared to 6-octadecyl-1,8-diazabicyclo[5.4.0]-7-undeceniumheptadecafluorooctanesulfonate of Comparative Example 1A, which was monoanion. It was assumed that the higher weight reduction temperatures and endothermic decomposition temperature were owing to the effect of dianion.

Moreover, it could be understood that the thermal stability was significantly improved compared to the commercially available perfluoropolyether Z-DOL (Comparative Example 2B) or Z-TETRAOL (Comparative Example 3B).

### Comparative Example 1B

### <Thermal Stability Measurement Results>

The 5% weight reduction temperature, 10% weight reduction temperature, and 20% weight reduction temperature, and the endothermic decomposition temperature of 6-octadecyl-1,8-diazabicyclo[5.4.0]-7-undeceniumheptadecafluorooctanesulfonate that was synthesized in Comparative Example 1A and was a monoanion ionic liquid were 361.9° C., 382.7° C., 403.5° C., and 417.2° C., respectively. Since the 6-octadecyl-1,8-diazabicyclo[5.4.0]-7-undeceniumheptadecafluorooctanesulfonate was the ionic liquid, the thermal stability was significantly higher than the commercially available perfluoropolyether.

### Comparative Example 2B

### <Thermal Stability Measurement Results>

Perfluoropolyether (Z-DOL), which was a commercial product, was generally used as a lubricant for magnetic recording media, has a hydroxyl group at a terminal, and has a molecular weight of about 2,000, was used as a lubricant of Comparative Example 2B. The 5% weight reduction temperature, 10% weight reduction temperature, and 20% weight reduction temperature of Z-DOL were 165.0° C., 197.0° C., and 226.0° C., respectively. The weight reduction was caused by evaporation.

### Comparative Example 3B

### <Thermal Stability Measurement Results>

Perfluoropolyether (Z-TETRAOL), which was a commercial product, was generally used as a lubricant for magnetic recording media, had several hydroxyl groups at terminals, and had a molecular weight of about 2,000, was used as a lubricant of Comparative Example 3B. The 5% weight reduction temperature, 10% weight reduction temperature, and 20% weight reduction temperature of Z-TETRAOL were 240.0° C., 261.0° C., and 282.0° C., respectively. Similarly to Z-DOL, the weight reduction was caused by evaporation.

The results of Examples 1B to 2B and Comparative Examples 1B to 3B are summarized in Table 2.

TABLE 2

| | | | | | | Endothermic |
|---|---|---|---|---|---|---|
| | | | Weight reduction | | | decomposition |
| | Synthesis | | temperature (° C.) | | | temperature |
| Example | Example | Compound name | 5% | 10% | 20% | (° C.) |
| Ex. 1B | Ex. 1A | 1,3-bis(1-octadecyl-2- | 347.7 | 368.3 | 387.9 | 400.5 |
| | | heptadecylimidazolium) | | | | |
| | | hexafluoropropane | | | | |
| | | disulfonate | | | | |
| Ex. 2B | Ex. 2A | 1,3-bis[6-octadecyl-1,8- | 362.0 | 383.1 | 407.1 | 421.5 |
| | | diazabicyclo[5.4.0]-7- | | | | |
| | | undecenium]hexafluoro | | | | |
| | | propanedisulfonate | | | | |
| Comp. | Comp. | 6-octadecyl-1,8-diaza | 361.9 | 382.7 | 403.5 | 417.2 |
| Ex. 1B | Ex. 1A | bicyclo[5.4.0]-7- | | | | |
| | | undeceniumheptadeca | | | | |
| | | fluorooctanesulfonate | | | | |
| Comp. | | Z-DOL | 165.0 | 197.0 | 226.0 | |
| Ex. 2B | | | | | | |
| Comp. | | Z-Tetraol | 240.0 | 261.0 | 282.0 | |
| Ex. 3B | | | | | | |

As described above, it could be understood that the ionic liquid-based lubricants had excellent thermal stability compared to the commercially available perfluoropolyether of Comparative Examples 2B and 3B.

Moreover, it was found from the comparison between Example 2B and Comparative Example 1B that the dianion ionic liquid having 6-octadecyl-1,8-diazabicyclo[5.4.0]-7-undecenium in the cation site had improved thermal stability, which demonstrated the effect of the dianion.

### Example 1C

### <Disk Durability Test 1>

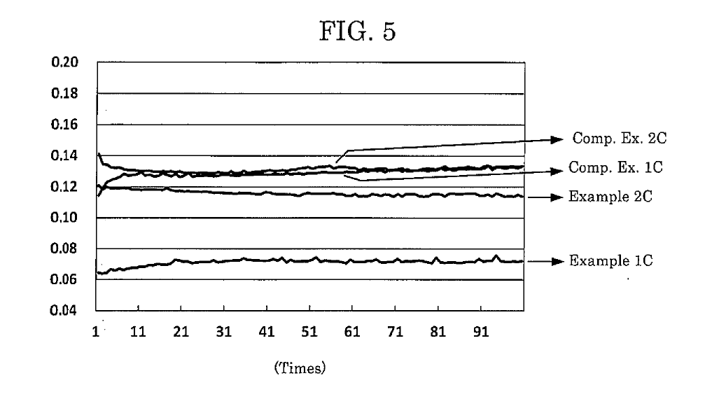

A friction test was performed on a lubricant including the ionic liquid of Example 1A by means of the pin-on-disk tester illustrated in FIG. 4. The result is presented in FIG. 5.

### Example 2C

### <Disk Durability Test 1>

A friction test was performed on a lubricant including the ionic liquid of Example 2A in the same manner as in Example 1C. The result is presented in FIG. 5.

### Comparative Example 1C

### <Disk Durability Test 1>

A friction test was performed on a lubricant including the ionic liquid of Comparative Example 1A in the same manner as in Example 1C. The result is presented in FIG. 5.

### Comparative Example 2C

### <Disk Durability Test 1>

A friction test was performed on Z-DOL that was a commercially available lubricant in the same manner as in Example 1C. The result is presented in FIG. 5.

As demonstrated in Comparative Example 2C, the friction coefficient of Z-DOL that was the commercially available lubricant was stably low in the pin-on-disk test. Moreover, the friction coefficient was stably low also in Comparative Example 1C because the ionic liquid-based lubricant was used.

As demonstrated in Example 1C, however, the friction coefficient of 1,3-bis(1-octadecyl-2-heptadecylimidazolium)hexafluoropropanedisulfonate that was the dianion lubricant was stable and the lowest in the tested examples, and was lower than the commercial product Z-DOL in Comparative Example 2C among the results of the return sliding of 100 times.

As demonstrated in Example 2C, moreover, the friction coefficient of 1,3-bis[6-octadecyl-1,8-diazabicyclo[5.4.0]-7-undecenium]hexafluoropropanedisulfonate was also lower than the commercially available Z-DOL in Comparative Example 2C among the results of the return sliding of 100 times. Moreover, the friction coefficient was lower compared to the monoanion demonstrated in Comparative Example 1C, which demonstrated the effect of dianion.

As described above, the friction coefficient of the ionic lubricant that originally had a low friction coefficient could be further reduced by using dianion, and the friction coefficient could be made lower than the friction coefficients of the commercial products.

The friction coefficients after 100 times are summarized in Table 3.

TABLE 3

| | | Friction coefficient |
|---|---|---|
| | Sample | after 100 times |
| Ex. 1C | Ex. 1A | 0.072 |
| Ex. 2C | Ex. 2A | 0.114 |
| Comp. | Comp. | 0.133 |
| Ex. 1C | Ex. 1A | |
| Comp. | Z-DOL | 0.133 |
| Ex. 2C | | |

### Example 1D

### <Disk Durability Test 2>

The above-described magnetic disk was produced using a lubricant including the ionic liquid of Example 1A. As presented in Table 4, the CSS measurement of the magnetic disk was greater than 50,000 times, and the CSS measurements after the heat tests of 300° C. and 350° C. were also greater than 50,000 times, hence excellent durability was exhibited.

### Example 2D

### <Disk Durability Test 2>

The above-described magnetic disk was produced using a lubricant including the ionic liquid of Example 2A. As presented in Table 4, the CSS measurement of the magnetic disk was greater than 50,000 times, and the CSS measurements after the heat tests of 300° C. and 350° C. were also greater than 50,000 times, hence excellent durability was exhibited.

### Comparative Example 1D

### <Disk Durability Test 2>

The above-described magnetic disk was produced using a lubricant including the ionic liquid of Comparative Example 1A. As presented in Table 4, the CSS measurement of the magnetic disk was greater than 50,000 times, the CSS measurement after the heat test of 300° C. was also greater than 50,000 times, and excellent durability was exhibited. However, the durability was deteriorated after heating at 350° C.

### Comparative Example 2D

### <Disk Durability Test 2>

The above-described magnetic disk was produced using a lubricant including Z-DOL. As presented in Table 4, the CSS measurement of the magnetic disk was greater than 50,000 times, but the CSS measurement after the heat test of 300° C. was 12,000 times and the durability was significantly deteriorated by the heating test of 350° C.

### Comparative Example 3D

### <Disk Durability Test 2>

The above-described magnetic disk was produced using a lubricant including Z-TETRAOL. As presented in Table 4, the CSS measurement of the magnetic disk was greater than 50,000 times, but the CSS measurement after the heat test of 300° C. was 36,000 times and the durability was significantly deteriorated by the heating test of 350° C.

The results of Examples 1D to 2D and Comparative Examples 1D to 3D are summarized in Table 4.

TABLE 4

| | | | | CSS durability | | CSS durability | |
|---|---|---|---|---|---|---|---|
| | | | | after heating at | | after heating at | |
| | Lubricant | CSS durability | | 300° C. | | 350° C. | |
| Ex. 1D | Ex. 1A | 25° C., | >50,000 | 25° C., | >50,000 | 25° C., | >50,000 |
| | | 60% RH | | 60% RH | | 60% RH | |
| Ex. 2D | Ex. 2A | 25° C., | >50,000 | 25° C., | >50,000 | 25° C., | >50,000 |
| | | 60% RH | | 60% RH | | 60% RH | |
| Comp. | Comp. | 25° C., | >50,000 | 25° C., | >50,000 | 25° C., | 36,000 |
| Ex. 1D | Ex. 1A | 60% RH | | 60% RH | | 60% RH | |
| Comp. | Z-DOL | 25° C., | >50,000 | 25° C., | 12,000 | 25° C., | 1,500 |
| Ex. 2D | | 60% RH | | 60% RH | | 60% RH | |
| Comp. | Z-Tetraol | 25° C., | >50,000 | 25° C., | 36,000 | 25° C., | 2,200 |
| Ex. 3D | | 60% RH | | 60% RH | | 60% RH | |

Next, examples where lubricants including novel dianion-based ionic liquids of Examples 1A to 2A, a lubricant of the monoanion-based ionic liquid of Comparative Example 1A, and Z-DOL and Z-TETRAOL of the commercial products are applied for magnetic tapes will be described.

### Examples 1E to 2E and Comparative Examples 1E to 3E

After producing magnetic tapes using lubricants including the ionic liquids of Examples 1A to 2A, the ionic liquid of Comparative Example 1A, Z-DOL, and Z-TETRAOL, respectively. Then, the following measurements were performed. The results are presented in Table 5.

Coefficient of friction of magnetic tape after shuttle run of 100 times:

In the environment having a temperature of −5° C., or in the environment having a temperature of 40° C. and relative humidity of 90%.

Still durability test:
In the environment having a temperature of −5° C., or in the environment having a temperature of 40° C. and relative humidity of 30%.

Shuttle durability test:

In the environment having a temperature of −5° C., or in the environment having a temperature of 40° C. and relative humidity of 90%.

Coefficient of friction of magnetic tape after shuttle run of 100 times after heating test:

In the environment having a temperature of −5° C., or in the environment having a temperature of 40° C. and relative humidity of 90%.

Still durability test after heating test:

In the environment having a temperature of −5° C., or in the environment having a temperature of 40° C. and relative humidity of 30%.

Shuttle durability test after heating test:

In the environment having a temperature of −5° C., or in the environment having a temperature of 40° C. and relative humidity of 90%.

The results of Examples 1E to 2E and Comparative Examples 1E to 3E are summarized in Table 5.

TABLE 5

| | | | | | | | | Friction | | Still | | Shuttle | |
|---|---|---|---|---|---|---|---|---|---|---|---|---|---|
| | | Friction | | Still | | Shuttle | | coefficient | | durability | | durability | |
| | | coefficient | | durability/ | | durability/ | | after 100 runs | | after heating/ | | after heating/ | |
| | Compound | after 100 runs | | min | | times | | after heating | | min | | times | |
| Ex. 1E | Ex. 1A | −5° C. | 0.15 | −5° C. | >60 | −5° C. | >200 | −5° C. | 0.15 | −5° C. | >60 | −5° C. | >200 |
| | | 40° C., | 0.16 | 40° C., | >60 | 40° C., | >200 | 40° C., | 0.16 | 40° C., | >60 | 40° C., | >200 |
| | | 90% | | 30% | | 90% | | 90% | | 30% | | 90% | |
| | | RH | | RH | | RH | | RH | | RH | | RH | |
| Ex. 2E | Ex. 2A | −5° C. | 0.18 | −5° C. | >60 | −5° C. | >200 | −5° C. | 0.19 | −5° C. | >60 | −5° C. | >200 |
| | | 40° C., | 0.19 | 40° C., | >60 | 40° C., | >200 | 40° C., | 0.19 | 40° C., | >60 | 40° C., | >200 |
| | | 90% | | 30% | | 90% | | 90% | | 30% | | 90% | |
| | | RH | | RH | | RH | | RH | | RH | | RH | |
| Comp. | Comp. | −5° C. | 0.22 | −5° C. | >60 | −5° C. | >200 | −5° C. | 0.23 | −5° C. | >60 | −5° C. | >200 |
| Ex. 1E | Ex. 1A | 40° C., | 0.23 | 40° C., | >60 | 40° C., | >200 | 40° C., | 0.24 | 40° C., | >60 | 40° C., | >200 |
| | | 90% | | 30% | | 90% | | 90% | | 30% | | 90% | |
| | | RH | | RH | | RH | | RH | | RH | | RH | |
| Comp. | Z-DOL | −5° C. | 0.25 | −5° C. | 12 | −5° C. | 59 | −5° C. | 0.32 | −5° C. | 12 | −5° C. | 46 |
| Ex. 2E | | 40° C., | 0.30 | 40° C., | 48 | 40° C., | 124 | 40° C., | 0.35 | 40° C., | 15 | 40° C., | 58 |
| | | 90% | | 30% | | 90% | | 90% | | 30% | | 90% | |
| | | RH | | RH | | RH | | RH | | RH | | RH | |
| Comp. | Z-TETRAOL | −5° C. | 0.22 | −5° C. | 25 | −5° C. | 65 | −5° C. | 0.28 | −5° C. | 23 | −5° C. | 55 |
| Ex. 3E | | 40° C., | 0.26 | 40° C., | 35 | 40° C., | 156 | 40° C., | 0.32 | 40° C., | 31 | 40° C., | 126 |
| | | 90% | | 30% | | 90% | | 90% | | 30% | | 90% | |
| | | RH | | RH | | RH | | RH | | RH | | RH | |

In Table 5, “>60” of the still durability denotes greater than 60 minutes.

In Table 5, “>200” of the shuttle durability denotes greater than 200 times.

The following facts were confirmed.

It was found that the magnetic tapes to which the lubricants including the ionic liquids of Examples 1A to 2A were applied had excellent friction properties, still durability, and shuttle durability.

It was found that the magnetic tape to which the lubricant including the ionic liquid of Comparative Example 1A was applied had excellent friction properties, still durability, and shuttle durability. It was exhibited that the lubricant of Comparative Example 1A gave excellent magnetic tape durability.

It was found that the magnetic tape to which Z-DOL was applied had a significant deterioration in still durability and shuttle durability.

It was found that the magnetic tape to which Z-TETRAOL was applied had a significant deterioration in still durability and shuttle durability.

It was found from Tables 2, 3, 4, and 5 that excellent heat resistance and pin-on-disk resistance, and excellent durability of a magnetic tape and magnetic disk could be obtained by using a lubricant of an ionic liquid that included a conjugate acid and a conjugate base having 2 or more anions in a molecule of the conjugate base, where a pKa of an acid that was a source of the conjugate base in acetonitrile was 10 or less.

As it was clear from the descriptions above, the lubricant of the ionic liquid that included a conjugate acid and a conjugate base having 2 or more anions in a molecule of the conjugate base, where a pKa of an acid that was a source of the conjugate base in acetonitrile was 10 or less had high decomposition temperature, and 5%, 10%, and 20% weight reduction temperatures, and had excellent thermal stability. It was found that use of the conjugate base having 2 or more anions in the molecule realized excellent heat resistance and friction resistance, particularly excellent durability after heating, compared to monoanion. Moreover, the lubricant could maintain excellent lubricity even under high temperature conditions compared to conventional perfluoropolyether, and lubricity could be maintained over a long period. Accordingly, a magnetic recording medium using the lubricant including the above-described ionic liquid could obtain extremely excellent running performances, abrasion resistance, and durability.

### REFERENCE SINGS LIST

11: substrate
12: base layer
13: magnetic layer
14: carbon protective layer
15: lubricant layer
21: substrate
22: magnetic layer
23: carbon protective layer
24: lubricant layer
25: back-coating layer

1. A lubricant comprising:
an ionic liquid including a conjugate acid and a conjugate base having 2 or more anions in a molecule of the conjugate base,
wherein the conjugate acid has a group including a straight-chain hydrocarbon group having 6 or more carbon atoms, and
a pKa of an acid that is a source of the conjugate base in acetonitrile is 10 or less.

2. The lubricant according to claim 1,
wherein the ionic liquid is represented by General Formula (1) below, 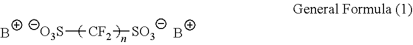
where, in General Formula (1), B⁺ is the conjugate acid and n is 1 or greater but 15 or less.

3. The lubricant according to claim 1,
wherein the conjugate acid is represented by General Formula (A) below, 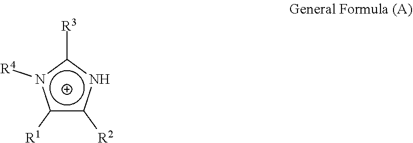
where, in General Formula (A), R¹ and R² are each a hydrogen atom or R¹ and R² may form a benzene ring together with carbon atoms to which R¹ and R² are bonded; R³ is a group including a straight-chain hydrocarbon group having 6 or more carbon atoms; and R⁴ is a hydrogen atom or a hydrocarbon group.

4. The lubricant according to claim 1,
wherein the conjugate acid is represented by General Formula (B), 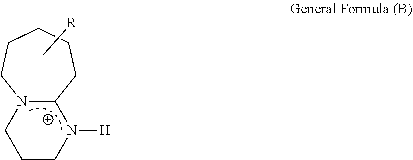
where, in General Formula (B), R is a group bonded to a bicyclic ring and including a straight-chain hydrocarbon group having 6 or more carbon atoms.

5. A magnetic recording medium comprising:
a non-magnetic support;
a magnetic layer disposed on the non-magnetic support; and
the lubricant according to claim 1, disposed on the magnetic layer.

6. An ionic liquid comprising:
a conjugate acid; and
a conjugate base having 2 or more anions in a molecule of the conjugate base,
wherein the conjugate acid has a group including a straight-chain hydrocarbon group having 6 or more carbon atoms, and
a pKa of an acid that is a source of the conjugate base in acetonitrile is 10 or less.

7. The ionic liquid according to claim 6,
wherein the ionic liquid is represented by General Formula (1) below, 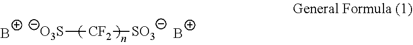
where, in General Formula (1), B⁺ is the conjugate acid and n is 1 or greater but 15 or less.

8. The ionic liquid according to claim 6,
wherein the conjugate acid is represented by General Formula (A) below, 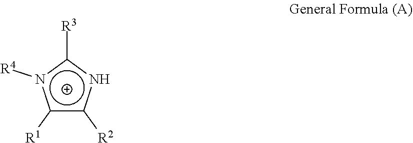
where, in General Formula (A), R¹ and R² are each a hydrogen atom or R¹ and R² may form a benzene ring together with carbon atoms to which R¹ and R² are bonded; R³ is a group including a straight-chain hydrocarbon group having 6 or more carbon atoms; and R⁴ is a hydrogen atom or a hydrocarbon group.

9. The ionic liquid according to claim 6,
wherein the conjugate acid is represented by General Formula (B), 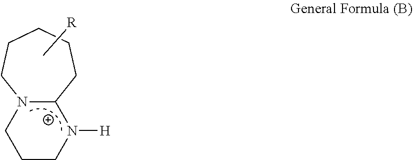
where, in General Formula (B), R is a group bonded to a bicyclic ring and including a straight-chain hydrocarbon group having 6 or more carbon atoms.
